(19) United States
(12) Patent Application Publication
(10) Pub. No.: US 2012/0141386 A1
(43) Pub. Date: Jun. 7, 2012

(54) Application of Antimicrobial and Glycemic Control Activities of Lo Han Kuo Fruit (Siraitia grosvenorii)

(75) Inventors: Chifu Huang, Lexington, KY (US); Jeffrey Lee Ebersole, Lexington, KY (US)

(73) Assignee: ORACEUTICALS, INC., Lexington, KY (US)

(21) Appl. No.: 12/959,163

(22) Filed: Dec. 2, 2010

(51) Int. Cl. A23L 1/221; A23C 23/00; A23G 3/38; A23G 9/32; A61P 31/10; A61K 8/97; A61Q 11/00; A61P 1/02; A61P 31/04; A23G 4/06; A61K 9/68

(52) U.S. Cl. 424/48; 426/3; 426/548; 424/58

(57) ABSTRACT

The present invention discloses an application of the extract of Lo Han Kuo (LHK) fruit (Siraitia grosvenorii Swingle) for its dual related functions. Specifically, this is related to the use of LHK extract for its antimicrobial activities against pathogens Streptococcus mutans, Porphyromonas gingivalis, A. actinomycecomitan, F. nucleatum, and Candida albicans, and its low glycemic index for use by general population as well as the diabetics to improve oral health.

### FIELD OF THE INVENTION

The present invention relates to Lo Han Kuo (LHK) fruit (Siraitia grosvenorii Swingle) extract and its components for the antimicrobial activities against oral pathogens, Streptococcus mutans, Porphyromonas gingivalis, A. actinomycecomitan, F. nucleatum, and Candida albicans, and the application in the prevention and treatment of oral diseases in general population, as well as the diabetic patients due to its low glycemic effect.

### BACKGROUND OF THE INVENTION

Two chronic diseases, periodontitis and type II diabetes mellitus, affect large populations. In a recent world-wide survey, it was reported that 80% of the world population in both developed and undeveloped countries suffer some type of oral disease. Besides individual hygiene procedures, diet composition contributes significantly to health/disease of the oral cavity. Dietary sucrose is a principal contributor to induce carious lesions caused by the oral pathogen Streptococcus mutans, which metabolizes sucrose into lactic acid, creating an acidic environment that demineralizes the enamel tooth surfaces, and extracellular polysaccharides that enhance accumulation of bacterial biofilms on the tooth surface. As refined sugar, and high fructose corn syrup (i.e. also leads to high acid production) are major staples in a broad array of processed foods, they also appear to be important contributors to the current “epidemic” of obesity and diabetes in the U.S.

A continuous effort has been invested supporting research and development to identify sweetening agents that can be placed in many types of foods under a wide range of conditions (e.g. heat, cold, acid, etc.) that may help limit the prevalence overweight and obese individuals, as well as being non-cariogenic. Various sugar alcohols, i.e. xylitol, sorbitol, have been included in a limited range of foodstuffs with the goal of sweetening without enhancing dental caries activity. However, not only are these substances considered high calorie, but also carry warnings of potential gastrointestinal disturbances in some individuals. Other no-calorie non-nutritive sweeteners, i.e. aspartame, saccharin, do not support growth or acid production by S. mutans, although they are often combined with materials that will enable growth/acid production by this microorganism (e.g. dextrose, maltodextrin), and also have some health concerns for subgroups in the population. Furthermore, limited information is available evaluating the ability of these sweeteners to inhibit S. mutans functions. Therefore, there remains an opportunity to identify a high intensive sweetening agent with desirable health benefits, such as prevention of dental caries through specific antibacterial properties, no-calorie levels related to obesity, and a low glycemic index to help manage blood sugar levels in diabetic patients. Application of this type of agent would constitute a benefit to the general population and will have a great market potential, as an agent that can be used widely in foods, beverages, mouthwashes, liquid medicines, and even coating for oral chewable medications.

Siraitia grosvenorii Swingle (also known as Lo Han Kuo fruit in China), a sweet fruit native to southern China, has been used for centuries as a traditional medicine for the treating dry cough, sore throat, diabetics, and constipation. The fruit extract is mainly composed of triterpene glycosides, also known as mogrosides, which have high intensive sweetness, low caloric content, and could serve as a substitute for sugar, safe for obese and diabetic patients to consume. The mogrosides are a group of compounds primarily consisting of mogrosides I-VI with variations in the position and the number of the sugar moieties attached to a mogrol group. For example, mogroside I has one sugar moiety, and mogroside II has two sugar moieties. Mogroside V has five sugar moieties and is the sweetest among the mogrosides, about 500 times sweeter than sucrose. Mogroside V is the predominant mogroside in the ripe fruit. Besides the sweetness, S. grosvenorii fruit extract possesses a wide range of pharmacologic and health-promoting properties including anti-tumor, anti-inflammatory, insulin-stimulating, and anti-oxidative activities. The dual antibacterial and insulin-stimulating activities of the bioactive components of Lo Han Kuo fruit have not been utilized. The LHK extract could be an attractive consumer sweetener as it could improve the oral health in both normal subjects and diabetic patients due to its dual bioactivities. This application utilizes the antimicrobial activity and the insulin-stimulating activity of mogrosides. This includes mogroside I, II, III, IV, V, mogroside VI, 11-o-Mogroside V, and Siamenoside I, that were evaluated for the possible role of the functional groups in these antimicrobial and insulin secretion activities.

### SUMMARY OF THE INVENTION

The purpose of the present invention is to provide a novel application of Lo Han Kuo extract, to ensure the full exploitation of the antimicrobial activity for human oral health. The characteristics are the discovery of anti-microbial activity of its bioactive compounds include mogrosides, mogroside I, II, III, IV, V and VI, 11-o-Mogroside V and Siamenoside I, siraitiflavandiol, rhamnocitrin-3-O-rhamnoside, and kaempferitrin against oral bacteria and their application for oral diseases. Specifically, the bioactive compounds include mogrosides, 11-o-Mogroside V and Siamenoside I, siraitiflavandiol, rhamnocitrin-3-O-rhamnoside, and kaempferitrin, have significant anti-microbial activity, allowing them to be applied to the prevention and treatment of oral diseases, such as caries, periodontitis, and gingivitis.

Moreover, Lo Han Kuo extract is a high intensive sweetening agent with desirable health benefits, such as prevention of dental caries through specific antibacterial properties, no-calorie levels related to obesity, and a low glycemic index to help manage blood sugar levels in diabetic patients. Application of this type of agent would constitute a benefit to the general population and will have a great market potential, as an agent that can be used widely in foods, beverages, mouthwashes, liquid medicines, and even coating for oral chewable medications. This application utilizes the antimicrobial activity and the insulin-stimulating activity of mogrosides. This includes mogroside I, II, III, IV, V, mogroside VI, 11-o-Mogroside V, and Siamenoside I, siraitiflavandiol, rhamnocitrin-3-O-rhamnoside, and kaempferitrin that are the functional groups in these antimicrobial and insulin secretion activities.

### DETAILED DESCRIPTION OF THE INVENTION

The following examples are presented by way of illustration, not of limitation.

### Example 1

Mogroside II, III, IV, V, VI, 11-o-Mogroside V, and Siamenoside I, siraitiflavandiol, rhamnocitrin-3-O-rhamnoside, and kaempferitrin were purified from LHK fruit were utilized. Oral bacteria Streptococcus mutans ATCC 25175 was grown in TSBYE media. Growth conditions were at 37° C. under anaerobic conditions (85% N2, 10% H2, and 5% CO2) for S. mutans. Various concentrations of mogrosides (25 μM, 125 μM, and 500 μM) were tested for antibacterial and antifungal activities in 96-well plates with wells containing 200 μl of TSBYE and a 10% inoculum of bacteria from an overnight culture. The above culture mixtures were diluted 1×10⁵ times and 10 μl of the diluents was each plated on blood-agar plates. The plates were incubated for 16-18 hours at 37° C. under anaerobic conditions. The colony forming units were counted afterwards. Significant antimicrobial activities of the mogrosides were observed.

### Example 2

RIN-5F cells (rat insulinoma) were obtained from ATCC (CRL-2058) and cultured in RPMI 1640 (10 mM glucose) supplemented with 10% (v/v) fetal calf serum, 2 mM L-glutamine, 1 mM sodium pyruvate, 100 IU/ml penicillin, 100 μg/ml streptomycin at 37° C., and 5% CO2. RIN-5F cells (1.5×106) were cultured in 75-cm2 tissue culture flasks and fed every 3 days. Cells were subcultured and seeded into 96-well plates with each well having RIN-5F cell density of 2.5×105. To determine the effects of the mogrosides on insulin secretion, the rat insulinoma cells were incubated with various concentrations of mogroside II, III, IV, V, VI, 11-o-Mogroside V, and Siamenoside I, siraitiflavandiol, rhamnocitrin-3-O-rhamnoside, and kaempferitrin, and absence of D-glucose as a negative control. Briefly, RIN-5F cells were grown for 14 hours on 1 mM D-glucose and then pre-incubated with KRB (Krebs-Ringer Bicarbonate) buffer (1.2 mM MgSO4, 4.7 mM KCl, 115 mM NaCl, 1.2 mM KH2PO4, 1.28 mM CaCl2, 24 mM NaHCO3, 10 mM HEPE-free acid, 3.3 mM Glucose, 0.1% BSA, pH 7.4) for 2 hours, followed by treatment with 1 mM or 30 mM D-glucose as a positive control and with the various concentrations of mogroside II, III, IV, V, VI, 11-o-Mogroside V, and Siamenoside I, for 1 hour. Aliquots of the media were collected and used to measure secreted insulin levels with a rat insulin EIA assay (ALPCO, Salem N.H.). Data are presented as the means of insulin concentration of two independent experiments, each with duplicate determinations. Statistical determinations were determined using a Student t test compared to control cultures and significant bioactivity was detected.

### Example 3

The concentration of 500 μM of mogroside II, III, IV, V, VI, 11-o-Mogroside V, and Siamenoside I, siraitiflavandiol, rhamnocitrin-3-O-rhamnoside, and kaempferitrin were used to test the inhibition activity against the growth of oral bacteria, A. actinomycetemcomitans and P. gingivalis. The experimental data demonstrated that there was significant difference in antibacterial activity among mogroside II, III, IV, V, VI, 11-o-Mogroside V, and Siganamse I against A. actinomycetemcomitans. Against A. actinomycetemcomitans, mogroside IV, V, VI, 11-o-Mogroside V showed higher antimicrobial activity than mogroside II and III, with mogroside V having the highest activity. Against P. gingivalis, mogroside II, IV, V, VI, 11-o-Mogroside V showed higher antimicrobial activity than mogroside III, with mogroside V having higher activity. Siraitiflavandiol, rhamnocitrin-3-O-rhamnoside, and kaempferitrin have the highest activity.

### Example 4

Mogroside II, III, IV, V, VI, 11-o-Mogroside V, and Siamenoside I were also evaluated in vitro for inhibition of growth of C. albicans. The experimental data demonstrated that there was significant difference in antimicrobial activity among mogroside II, III, IV, V, VI, 11-o-Mogroside V, and Siamenoside I against C. albicans. Against C. albicans, mogroside II, III, IV, V, and 11-o-Mogroside V showed higher antimicrobial activity than mogroside VI and Siamenoside I. C. albicans is the principal fungal species that colonizes the human oral cavity. Siraitiflavandiol, rhamnocitrin-3-O-rhamnoside, and kaempferitrin also have good activity. Thus, the ability to identify agents that demonstrate some specificity as anti-fungal agents, and that could be incorporated into medicaments that would be delivered to oral mucosal sites, would broaden the capacity to prevent and treat the range of oral infections.

### Example 5

The weight percentage composition of antimicrobial oral chewing tablet, containing Lo Han Kuo extract/mogroside, is as follows:

| | |
|---|---|
| Lo Han Kuo extract | 1.0% |
| Mannitol/or xylitol or sorbitol | 5.0% |
| Dextrin | 18.5% |
| Menthol | 0.5% |
| HPMC | 4.0% |
| Manesium stearate | 0.5% |
| Other ingredients | 80.0% |

### Example 6

The weight percentage composition of chewing gum, containing LHK extract/mogroside is as follows:

| | |
|---|---|
| Lo Han Kuo extract | 1-5% |
| Gum base | 80.0% |
| Mannitol, or xylitol or sorbitol | 10.0% |
| Menthol | 0.2% |
| Lecithin | 0.6% |
| Edible flavors | 0.9% |
| (such as banana or other flavors) | |
| Edible citric acid | 0.2% |

### Example 7

The weight percentage composition of chewing gum, containing fish oil fatty acids, eicosapentaenoic acid (EPA), docosahexaenoic Acid (DHA), α-linolenic acid (ALA), (EPA, or DHA, or ALA or ARA or GLA)/or fatty acid methyl esters/or fatty acid ethyl esters, is as follows:

| | |
|---|---|
| Lo Han Kuo extract | 1% |
| Omega-3/omega-6 fatty acids/or | 1-5% |
| their methyl esters/or ethyl esters | |
| Gum base | 80.0% |
| High intense sweetener | 2% |
| mogroside, rebaudisoside | |
| Menthol | 0.2% |
| Lecithin | 0.6% |
| Edible flavors | 0.9% |
| (such as banana or other flavors) | |
| Edible citric acid | 0.2% |

### Example 8

The weight percentage composition of dairy products, containing mogrosides, omega-3 fatty acids (EPA, or DHA, or ALA) or omega-6 fatty acid (ARA or GLA), or their fatty acid methyl ester/or fish oil fatty acid ethyl ester, is as follows:

| | |
|---|---|
| Omega-3 fatty acid/or methyl | 1-5% |
| ester/or fish oil ethyl ester | |
| Milk | 90-95% |
| Lo Han Kuo extract | 1-5% |

### Example 9

The weight percentage composition of candy, containing omega-3 fatty acids (EPA, DHA, ALA), or omega-6 fatty acid (ARA or GLA),/or omega fatty acid methyl ester/ethyl esters, is as follows:

| | |
|---|---|
| Fish oil/or fish oil methyl ester/or | 1-5% |
| fish oil ethyl ester | |
| Milk powder | 5% |
| Lo Han Kuo extract | 1% |
| Dry starch syrup solid objects | 30~35% |
| Non-fat milk solids | 5~10% |
| Plant fat | 15-20% |
| Salt | 0.2-0.3% |
| Colloidal | 1.5-2.0% |

The preparation of the functional candy by weight of each component were made as: Lo Han Kuo extract (1%), omega fatty acids (EPA, or DHA, or ALA, or ARA, GLA)/or their methyl ester/or ethyl ester dissolved is about 0.1-10%. The functional candy can be used to prevent cavity, gum disease, periodontitis with its antimicrobial activity.

### Example 10

The weight percentage composition of toothpaste, containing omega fatty acids (EPA, or DHA, or ALA, or ARA, or GLA)/or omega fatty acid methyl/ethyl ester or fish oil fatty acid ethyl ester is as follows:

| | |
|---|---|
| Omega fatty acids/or omega fatty | 1-5% |
| acid methyl ester/or ethyl ester | |
| Baking soda | 35% |
| Lo Han Kuo extract | 1% |
| Hydrogen peroxide | 5~10% |
| Plant fat | 15-20% |
| Flavors | 1.5-2.0% |

The preparation of a therapeutic toothpaste by weight of each component would be made from fatty acids as the bioactive components, where the active content of omega-3 fatty acids (EPA, or DHA, or ALA, or ARA, GLA)/or their methyl ester/or ethyl ester dissolved at about 0.1-10%. The toothpaste can be used as an adjunct for prevention of dental caries, gingivitis, and periodontal diseases. Other ingredients include baking soda, glycerin, hydrogen peroxide and a trace amount of peppermint oil.

### Example 11

The preparation of a low-calorie ice cream with anti-caries properties would be made by weight of each component were calculated as, where active content of extract is about 0.1-10%. The ice cream could part of a healthy diet that also enhances control of bacteria in the mouth.

Omega fatty acids, or esters 0.01˜5%
½ cup low fat milk
1/10 tablespoon of Lo Han Kuo extract
¼ teaspoon of vanilla extract or cocoa
Prepare ice cream via standard protocol

### BEST MODE FOR CARRYING OUT THE INVENTION

The preferred embodiments of this invention are described herein, which includes the best mode that is known to the inventors for implementing the invention. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

1. The present invention relates to utilization of the Lo Han Kuo fruit extract for its health benefits. The application of the fruit extract is related to its dual functions as an antimicrobial therapeutic against Streptococcus mutans, Porphyromonas gingivalis, A. actinomycecomitan, F. nucleatum, and Candida albicans to improve oral health of human and animals.

2. The present invention relates to utilization of the Lo Han Kuo fruit extract for its health benefits. The application of the fruit extract is related to its low glycemic index enabling use by the general population, as well as diabetic patients.

3. As mentioned in claim 1 and claim 2, the application of the fruit extract is due to the dual activities of its bioactive components. The bioactive compounds include mogrosides, including mogroside I, II, III, IV, V and VI, 11-o-Mogroside V and Siamenoside I, for the prevention and treatment of oral infections in normal individuals and diabetic patients.

4. As mentioned in claim 1 and claim 2, the bioactive compounds also include siraitiflavandiol, rhamnocitrin-3-O-rhamnoside, and kaempferitrin, for the prevention and treatment of oral diseases in normal individuals and diabetic patients.

5. As mentioned in claim 1, the above compounds are effective against many oral pathogens, for example, Streptococcus mutans, Porphyromonas gingivalis, A. actinomycecomitan, F. nucleatum, and the yeast Candida albicans but not limited to these genera and species.

6. As mentioned in claim 2, the above compounds are effective in enhancing insulin production by pancreatic cells.

7. As mentioned in claim 1 and claim 2, the key use would be via ingestion including chewing gum, beverages, coffee, and other dairy products.

8. As mentioned in claim 1 and claim 2, the application of LHK extract for the prevention and treatment of oral infections, include dental caries, periodontitis, gingivitis, mouth ulcers, and halitosis, but not limited to.

9. As mentioned in claim 8, the role of oral treatment with these bioactive compounds can be used in any formulation, including tablets, sprays, powders, chewable/chewing gum, bioadhesive films or paints, ointments, gels, paste, mouthwash, or other solutions, etc. The tablets for oral administration include oral tablets, dispersible tablets, effervescent tablets, chewable/chewing tablets, chewable/chewing gums, but not limited to.
